(19) United States
(12) Patent Application Publication
(10) Pub. No.: US 2026/0283793 A1
(43) Pub. Date: Sep. 24, 2026

(54) DEVICE AND METHOD WITH REDUCED PACEMAKER RATE IN HEART VALVE REPLACEMENT

(71) Applicant: JenaValve Technology, Inc., Irvine, CA (US)

(72) Inventors: Stefan SCHRECK, San Clemente, CA (US); Hussain S. RANGWALA, Villa Park, CA (US); Payam SAFFARI, Aliso Viejo, CA (US)

(73) Assignee: JenaValve Technology, Inc., Irvine, CA (US)

(21) Appl. No.: 19/683,600

(22) Filed: May 20, 2026

Related U.S. Application Data
(63) Continuation of application No. 17/664,414, filed on May 22, 2022, now Pat. No. 12,636,148.
Application No. 17/664,414 is a continuation of application No. 16/926,585, filed on Jul. 10, 2020, now Pat. No. 11,337,800.
Application No. 16/926,585 is a continuation of application No. 15/570,897, filed on Apr. 18, 2016, now Pat. No. 10,709,555.
(60) Provisional application No. 62/155,849, filed on May 1, 2015.

(51) Int. Cl. A61F 2/24; A61L 31/18

(52) U.S. Cl. CPC A61F 2/2418; A61L 31/18; A61F 2210/0014; A61F 2230/0013; A61F 2230/0054; A61F 2230/0056; A61F 2230/006; A61F 2250/0032; A61F 2250/0097; A61F 2250/0098

(57) ABSTRACT

The disclosure relates to heart valve prostheses with the reduced need of pacemaker implantation and improved means for positioning the replacement heart valve.

### CROSS-REFERENCE TO RELATED APPLICATIONS

This application is a continuation of U.S. patent application Ser. No. 17/664,414, filed May 22, 2022, now U.S. Pat. No. 12,636,148, which is a continuation of U.S. patent application Ser. No. 16/926,585, filed Jul. 10, 2020, now U.S. Pat. No. 11,337,800, which is a continuation of U.S. patent application Ser. No. 15/570,897, filed Oct. 31, 2017, now U.S. Pat. No. 10,709,555, which is a national stage filing under 35 U.S.C. § 371 of International Application No. PCT/EP2016/058532, filed on Apr. 18, 2016, which published in the English language and claims the benefit of priority to U.S. Provisional Application No. 62/155,849, filed on May 1, 2015, the entire contents of each of which are incorporated herein by reference. This application is also related to U.S. patent application Ser. No. 18/634,703, filed Apr. 12, 2024, now U.S. Pat. No. 12,343,255, the entire contents of which are incorporated herein by reference.

### TECHNICAL FIELD

The present disclosure relates to heart valve prostheses with reduced pace maker rate and means and methods for visualization of the correct implantation of a medical device at the target site in a patient.

### BACKGROUND

A number of applications have been established making use of medical devices, which can be delivered by way of minimally invasive methods in a patient. An example of such a medical device is a heart valve prosthesis.

Various replacement heart valves for aortic, mitral and tricuspid heart valves are currently available. In particular in the context of aortic heart valves replacement valves a side effect is the necessity of pace maker implantation in many implantations and devices. The requirement of a pacemaker can be as high as 30% in state of the art device implantation. This does not only have the drawback of another surgery and medical device with all its negative implications for the patient but also imposes increased cost in the context of such a heart valve replacement therapy. Accordingly there is a need to avoid or at least reduce the rate of pacemakers in such treatments.

Another problem often occurs when trying to achieve a correct implantation and positioning of the prosthesis at the target site in order to fully and reliably exhibit the prosthesis'function.

A particular example is a catheter-based aortic valve prosthesis consisting of a self-expanding stent and a valve known for treating aortic insufficiency. Such heart valve prostheses are positioned at the aortic annulus to replace the endogenous aortic valve. The aim is to correctly position the heart valve prosthesis with regard to the aortic annulus and the endogenous cusps.

W02004/019825 describes an aortic prosthesis wherein the prosthesis exhibits feelers which are meant to be deployed first and placed into the aortic cusps. Once the feelers have been placed within the cusps the stent is deployed to complete the implantation. The entire implant procedure is guided by fluoroscopic imaging. The stent and feelers are visible under fluoroscopy. The aorta, aortic valve, and left ventricle are visualized indirectly by injecting contrast medium through an angiographic catheter into the left ventricle and ascending aorta. During valve deployment the angiographic catheters are retracted to avoid interference between the stent and the angiographic catheter. Thus, the operator mainly relies on tactile feedback for feeler placement.

In case of a transfemoral valve replacement, the tactile feedback may be inconsistent due to the tortuosity of the access vessels and the curvature of the aortic arch. As a result, the prosthesis may not be placed sufficiently correct at its target site. In case a prosthesis is applied that uses feelers, cusp positioners, hooks, rims or similar means to provide for precise positioning and/or targeting the endogenous leaflet cusps, these means may not be correctly positioned and/or they may be placed away from the base of the cusps or may damage the cusps.

There exists thus the need for better guiding the placement of the valve prosthesis and to avoid damage of the endogenous heart tissue and in particular cusp damage or perforation.

Another issue is leakage of blood between the replacement heart valve and the endogenous tissue e.g. at the annular ring of the aortic heart valve. Known prostheses try to improve leak tightness by applying or forming a ring or band along the annular ring and cover the prosthesis by a symmetric band made of biological or synthetic tissue. Some disclosures try to improve the leak tightness with the combination of the outward force of the prosthesis and the symmetrical band aligned along the annular ring. This approach is commonly used and it is acknowledged that a symmetrical sealing ring is a useful approach, which serves the purpose, however, this approach is not always 100% successful.

Yet another issue is the need for pacemaker implantation after heart valve replacement therapy. In currently available therapies and heart valve prostheses a considerable number of patients require a pacemaker implantation after heart valve replacement therapy. Currently there are a number of replacement heart valves on the market like the Sapiens HVT, the Lotus device, the Corevalve device or the Symetis device all for aortic heart valve replacement with a minimally invasive approach. The percentages of the requirement for pacemaker transplantation vary between these products. It is acknowledged that the pacemaker requirement is unwanted and makes yet another surgery necessary including all its negative implications.

Accordingly, there is a need to provide for methods and replacement heart valves with a reduced need of pacemaker implantation.

Hence it is one object to provide for a replacement heart valve therapy with reduced pacemaker rate.

It is another object to provide for a means and a method for safe positioning replacement heart valves into an individual's body at a target site.

It is yet another object to be able to visually control the correct positioning of a replacement heart valve.

It is another object to provide heart valve prostheses with good or even advantageous leak tightness features.

It is yet another object to provide for a replacement heart valve therapy wherein the replacement heart valve is engineered in a manner so as to reduce or even substantially avoid the disadvantages of the prior art, or to provide for a replacement heart valve that combines the advantages of being capable of secure and correct positioning and at the same time exhibiting a reduced need for pacemaker implantation.

It is yet another object to provide for a replacement heart valve prosthesis which has improved properties or/and which exhibits advantageous features with respect to the pacemaker need, e.g., a reduced pacemaker need vis-à-vis known devices or a pacemaker rate that is acceptable, easy positioning, and/or good leak tightness features.

### SUMMARY

In one aspect are disclosed replacement heart valve prostheses with the reduced need of pacemakers. The reduced need for pacemaker application after the replacement heart valve implantation according to some embodiments may be related to aspects of the prosthesis design.

In another aspect are disclosed methods for the minimally invasive application of said replacement heart valves by use of a catheter device in a transfemoral or transapical manner. The catheter may be adapted to the prosthesis in order to allow easy and correct implantation into the heart of an individual.

In another aspect is disclosed means for visualizing the positioning of replacement heart valves at an implant site inside an individual's body, wherein the medical implant exhibits a deformable indicator means.

In another aspect is disclosed a method for the visualization of the positioning of a medical implant exhibiting deformable detector means at an implant site inside a patient body wherein i. the implant is delivered by appropriate means close to or at least relatively close to the target implantation site; ii. the implant is approached to its final target site; iii. the approach of the implant is stopped when the deformable detector means indicate contact with the tissue of the final target site.

In yet another aspect is disclosed a method for minimally invasive implantation of a replacement heart valve in an individual.

### BRIEF DESCRIPTION OF THE DRAWINGS

The Figures will describe various aspects without being understood as restrictive. The skilled person will also appreciate that any of the features as described in the Figures or in any of the examples mentioned herein may be combined with any other features or a number of features as described throughout the specification and claims herein.

FIGS. 1a-1c, at the left side, (FIG. 1a) describe a top view of a heart with the bundle of His (1), the septum (2), the mitral valve (3), the aortic valve with right leaflet (4), left leaflet (LC) and non-coronary leaflet (6). In the middle (FIG. 1b) is depicted the annular ring (7) of the aortic valve and the aortic arch (8). On the right side (FIG. 1c) a prosthesis is placed at the site of the endogenous aortic heart valve in the annular ring (7).

FIG. 2 depicts an aortic replacement valve according to an embodiment of the disclosure (14) flapped open to show three sections (16), (17), (18) from left to right and the distal prosthesis area. (9), (10), and (11) refer to the right coronary sinus, non-coronary sinus and left coronary sinus, respectively. The left coronary (12) and right coronary (13) are shown wherein the prosthesis is designed so as not to cover the coronaries (12) and (13). The prosthesis (14) in this embodiment exhibits between section (16) and (18) an area (19) which has a higher proximal edge.

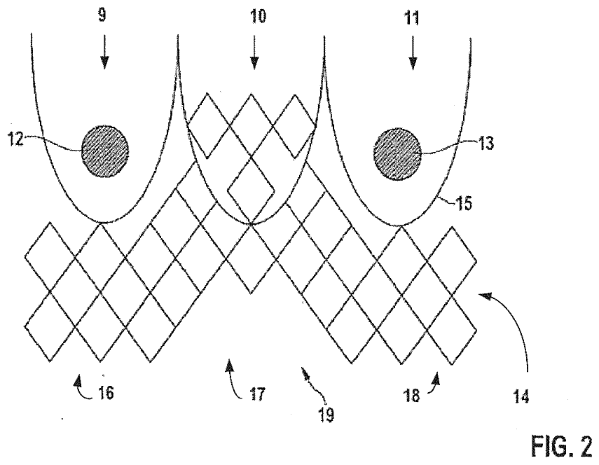

FIG. 3 illustrates a part of the prosthesis according to another exemplary embodiment of the disclosure (14) wherein particular aspects of the stent forming part of the prosthesis are depicted. Other parts as necessary may form part of a prosthesis of the present disclosure which are not explicitly shown but which may form part of the prosthesis as disclosed herein. In particular the locator (20) and the proximal stent ring (21) are depicted. The proximal end of locator (20) may be specifically designed and engineered to provide for a particular dimension and distance with regard to the proximal end of the prosthesis according to the disclosure (14). In the present illustration it is 6 mm. However, other dimensions can be useful depending on the particular needs and requirements which can be adapted to, such as, e.g., a range of between 1 and 10 mm, e.g. 4 mm, 5 mm, 7 mm, 8 mm.

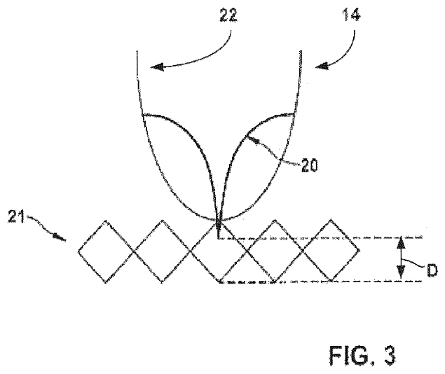

FIG. 4a (non-expanded state) and FIG. 4b (expanded state) show a prosthesis according to an exemplary embodiment (14) with a locator (20) attached to fastening arches (23). Also indicated are the proximal and distal prosthesis areas (21) and (22), respectively. The locator (20) may have an adapted design and positioning with respect to and as being connected with fastening arches (23). Also indicated is the foreshortening distance (24) and the fact that in its expanded state the locator(s) (20) may at least partially superpose some areas of the remaining prosthesis. The prosthesis (4) may exhibit three locators (20) but it may also be feasible to use more or less locators, e.g. two locators. Also the locators (20) may have the same design and length or may represent different embodiments, e.g., being engineered differently.

FIGS. 5a-5b show a variation of the prosthesis of FIGS. 4a-4b wherein the locator (20) is differently engineered and the foreshortening distance (24) is achieved in a variation of the one depicted in FIGS. 4a-4b, wherein FIGS. 4a-4b and 5a-5b show variations in the connection between the locator (20) and the fastening arches (23).

FIGS. 6a-6c show a prosthesis (14) according to another exemplary embodiment of the disclosure exhibiting a feeler and an indicator, and wherein the sequence of positioning of a medical implant according to the present disclosure is depicted, wherein FIG. 6a shows an aortic valve prosthesis comprising a stent, arch-shaped locator (20) connected to the commissures and extending proximally, and an arch-shaped indicator (25) connected to the commissures and extended proximally beyond the locators (20). The indicators may be formed e.g. from flexible radiopaque wires. FIG. 6b shows a locator (20) and corresponding indicator located distal to a native cusp. The indicator (25) is in its undistorted configuration. In FIG. 6c, the locator is advanced to the base of the native cusp. The indicator contacts the cusp first and is deformed by the force used to further advance the locator (20) and stent. The locator (20) is relatively stiff and does not deform. When the locator (20) approaches the base of the cusp, the most proximal segment of the indicator and the most segment of the locator (20) approach each other and contact each other. This configuration indicates that the locators are in full contact with the cusps. The locator and the indicator are radiopaque and their physical location to each other can be visualized using fluoroscopy. Alternatively, the locators (20) and/or the indicator may be made from non-radiopaque material. Individual radiopaque markers may be placed on the proximal segments of the locator (20) and indicator to visualize their respective location using fluoroscopy.

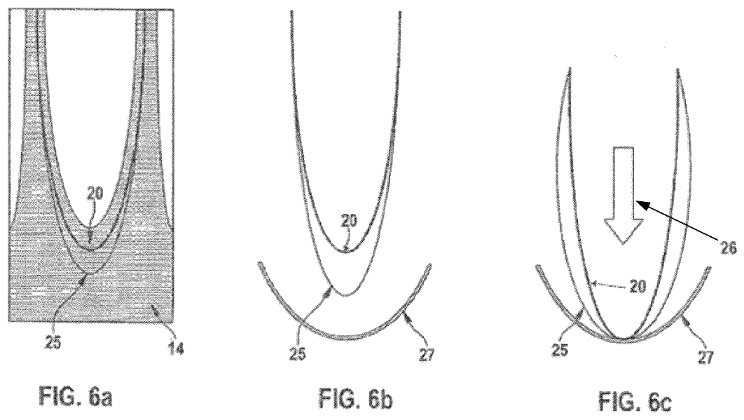

FIGS. 7a-7c show alternative embodiments of the locators (20) and radiopaque indicators (25). In FIGS. 7a-7c, a series of “antennas” extend from the proximal segment of the feeler in the direction of the cusps. The antennas may be made from flexible material such as a memory alloy. The antennas may have similar properties as guide wire tips to prevent tissue damage. The tip of the antenna or the entire antenna may be radiopaque. When the antennas contact the cusp (27) they are deflected. When multiple antennas are used, the array of antenna tips outlines the shape of the cusps (27). This may be helpful in visualizing the center of the cusp (27).

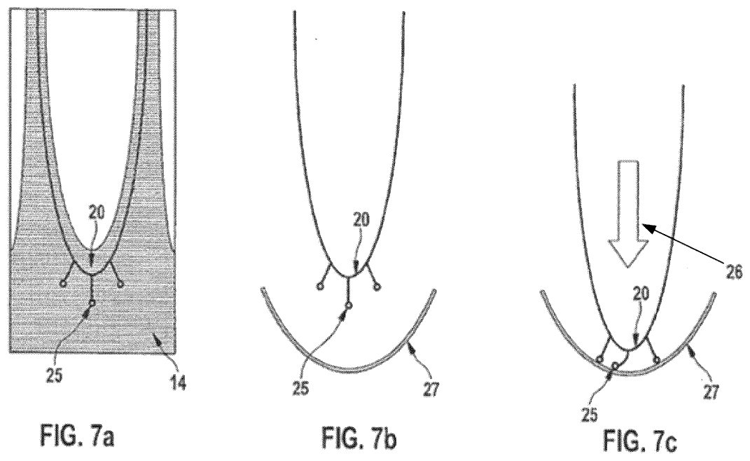

FIGS. 8a-8c show another alternative embodiment of the locators (20) and indicators (25). Each locator has an “M” shape with the end of the M being connected to the stent. The center “Y” segment of the locator sits inside the valve cusp sandwiching the cusp between the Y segment and the stent. A single indicator (25) is connected to the base of the Y shaped locator (20) segment. The indicator may be similar in shape and construction of the indicators in FIGS. 7a-7c. Alternatively, the indicator (25) may be made from soft fabric, textile, mammalian tissue, or a polymer. Polymers may include but not limited to silicone, polyurethane, and ePTFE. The fabric, textile, and mammalian tissue may be attached to the locators (20) by sutures, clips, staples, or adhesives. The polymer may be attached to the locators (20) by adhesives, heat fusion, or over-molding. At least the proximal end of the indicator (25) may be radiopaque. Radiopaque markers may be sewed into the fabric or imbedded or molded into the polymer material. The implantation direction into the cusps (27) is indicated by arrow (26).

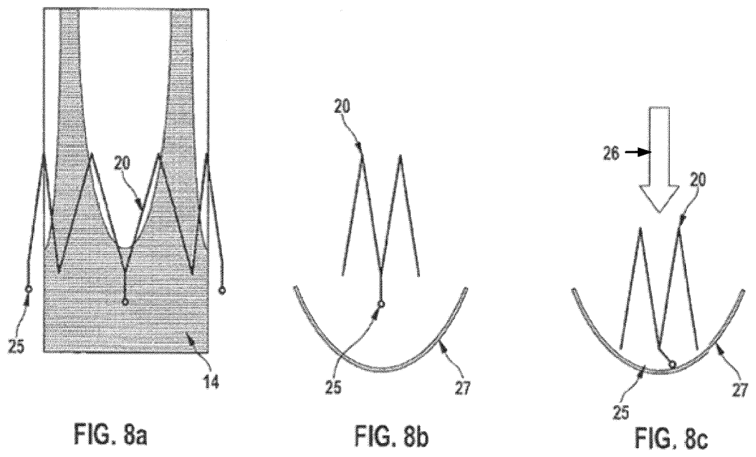

FIGS. 9a-9c illustrate an indicator in form of a locator cover (28) which is affixed to the locator (20) and comprises one or more radio-opaque means for visualization with respective means and methods known by the skilled person during surgery. The locator cover (28) can be cut in different forms and seizes as is appropriate for affixing same to the locator (20). Such a cover may be made of material compatible with the remaining components of the prosthesis and may exhibit biocompatible characteristics. The cover (28) may be well compatible with its function and long term implantation into an individual. The cover and as well the other components of the prosthesis such as stent, biological and non-biological materials can be covered or coated with a coating which may facilitate implantation and/or biocompatibility with the tissue of the implantation site in the heart. The FIGS. 9a, 9b, 9c illustrate variations in the cover (28) and the positioning of the radio-opaque markers (29).

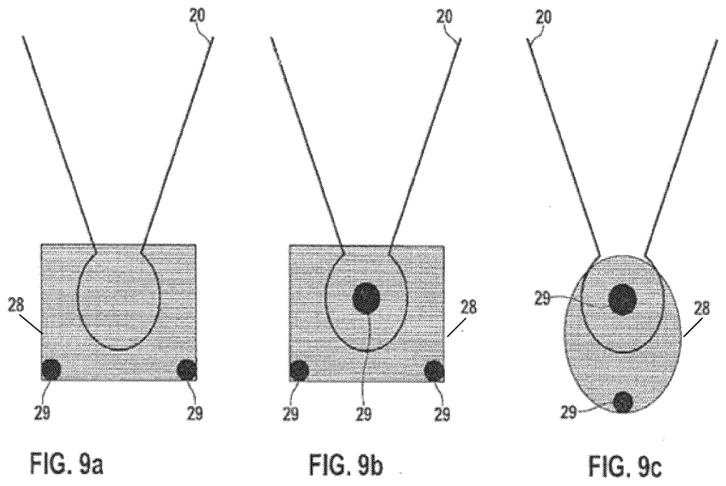

FIGS. 10a-10c show variations of locators (20) including a radio-opaque markers (29) wherein the locators (20) as well as the markers (29) are engineered with variations and additional variations in size, dimension, marker (29) location are well within the scope of the present disclosure.

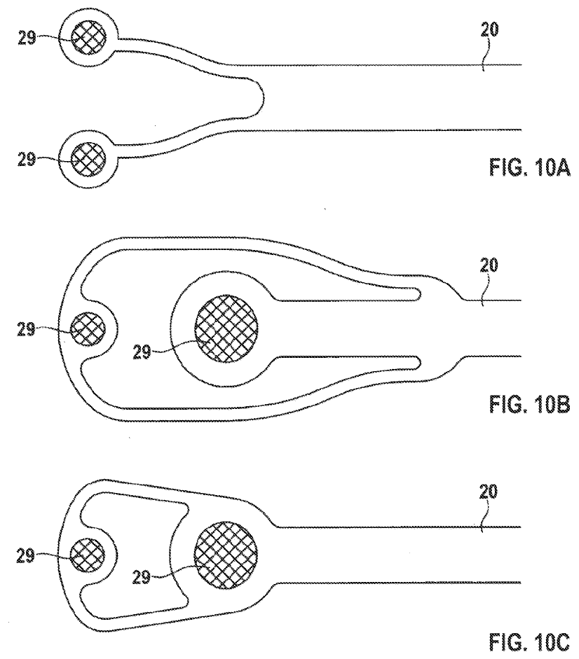

FIG. 11 depicts a prosthesis (14) according to an exemplary embodiment of the present disclosure and illustrates the cover (28) including the radio-opaque marker areas (29) attached to a locator (20) and indicates the distal area (22), the proximal area (21), and the fixation arches (23).

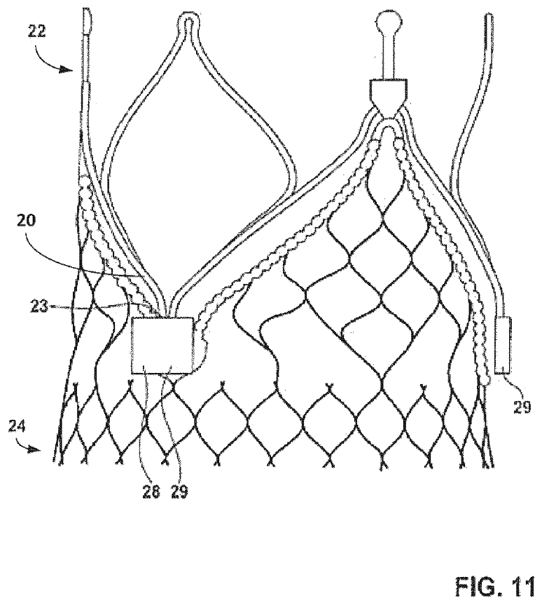

FIG. 12 shows the prosthesis (14) implanted at the aortic annulus ring and the use for facilitation of better positioning by way of the locators (20) and radio-opaque (29) marked indicator in form of a locator cover (28) wherein calcified leaflets (31), the sinus of vasalva (30) and the (32) are shown.

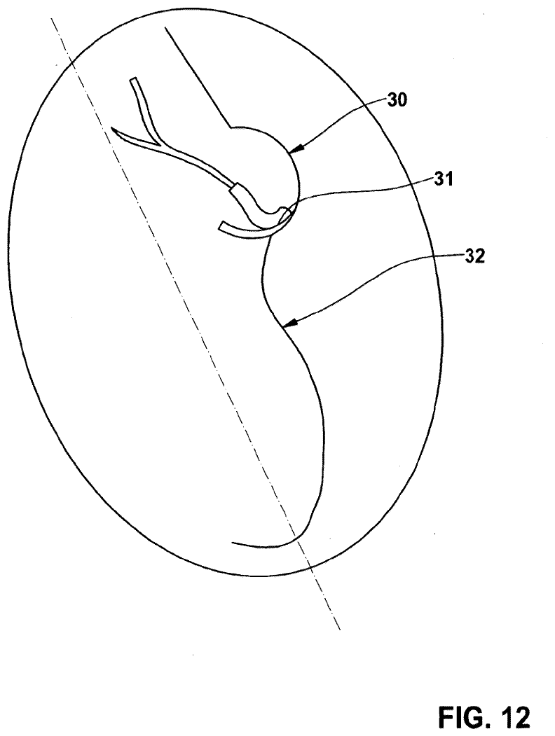

FIGS. 13a-13c show another way of illustrating the prosthesis (14) as of FIG. 12 positioned and implanted at its target site.

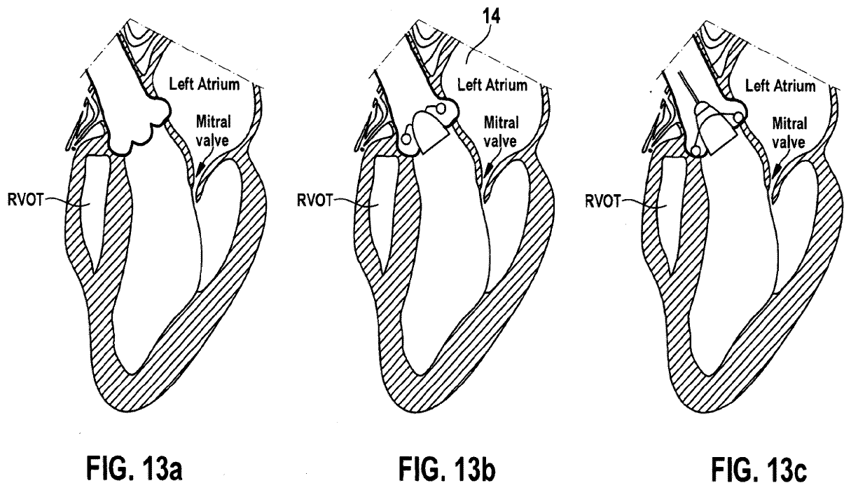

### DETAILED DESCRIPTION

The objects of the disclosure may be addressed by the prostheses and methods as disclosed herein.

In the following some terms of the disclosure will be defined and unless stated otherwise they will represent the meaning for the purpose of the description of the subject matter described herein.

“Heart valve prosthesis” or “prosthesis” or “medical implant” or “medical device” in the sense of the disclosure is any medical device like a heart valve that may be implanted into a patient by means of a minimally invasive procedure e.g. by way of the use of a catheter or a similar delivery device. “Prosthesis” relates to aortic, mitral and tricuspid replacement heart valves.

The term “proximal” refers to the part of the prosthesis which will be closer to the apex of the heart during or when implanted, and the term “distal” refers to the part of the prosthesis which is further away from the apex of the heart during or when implanted. The term “proximal” may also be used in the context of the locator means.

The term “varying” in connection with the proximal end or the edge of the proximal end refers to the specific design of the disclosed subject matter, wherein the edge of the proximal end of the prosthesis can be uniform and describe a ring ending at the same level. On the other hand different sections of the prosthesis can be designed in a way so as to have their edges of the proximal ends at differing levels and thus represent differing distances to e.g. the locator means in case the prosthesis' consists of three sections wherein each section comprises on locator means. Thus the proximal end of the prosthesis may exhibit an undulating proximal edge.

“Tube perimeter” refers to e.g. a nitinol tube which is laser cut in order to receive the stent component of the prosthesis and which describes the same inner and outer dimension and surface over the tube. Accordingly, in some embodiments, no parts of the cut stent may substantially stick inwardly or outwardly of said tube.

The term “foreshortening” describes the change of position or position of the proximal end of the locator means when the stent component is expanded and the proximal end of the locator means moves outwardly of the tube perimeter and towards the proximal end of the prosthesis. Thus the distance between the proximal end of the locator means and the proximal end of the stent may be reduced “shortened” as compared to the non-expanded position of the proximal end of the locator means. The “foreshortening” may depend on the design of the locator means as such and on the connection with other parts of the stent. Thus a design such as an arch may be advantageous and its connections with its ends at each side with a fastening arch of the stent. The skilled person will appreciate that in this manner the “foreshortening” can be defined and it can vary between 1 and 15 mm, or one can achieve a foreshortening of 1, 2, 3, 4, 5, 6, 7, 8, 9, 10, 11, 12, 13, 14, or 15 mm.

A “fastening arch” is a part of the stent to which the valve component is fixed, e.g., by suitable means and methods.

A “non-expanded state” and “expanded state” of the prosthesis refers to a design wherein the prosthesis can be crimped and placed to or in a catheter for minimally invasive delivery purposes. The prosthesis may be expanded by balloon expansion or self-expandable, and when placed and positioned at its target site it may exhibit its “expanded state”. Thus the “non-expanded state” represents the minimal diameter of the prosthesis and the “expanded state” represents its biggest diameter. It will be appreciated by the skilled person that the prosthesis when positioned at its target site may exhibit outward forces against e.g. the annular ring which may exhibit a force in counter direction and thus the prosthesis may not exhibit in this state its maximal diameter. The outward force may contribute to the fixation/positioning of the prosthesis.

“Heart pacemaker” or “pacemaker” in the sense of the disclosure are devices to trigger and control an appropriate or normal heart rate in an individual.

“Pacemaker rate” or the “need for pacemaker” or “the need for pacemaker implantation” relates to the fact that in heart valve replacement therapy after implantation of the prosthesis a number of patients require pacemakers. Accordingly, an additional surgery is required in such individuals.

“Pacemaker rate” in this context refers to the percentage of individuals who need a pacemaker after valve implant. The pacemaker rate in currently available replacement heart valve treatment is in the range of 10% to 30%.

“Indicator means” or “indicators” in the sense of the disclosure are any constructive means that allow or facilitate the easy and precise positioning, e.g., by way of a visualization apparatus or devices that allow controlling the position of the medical device within a patient.

“Locator means” or “locator” or “feeler” in the sense of the disclosure is to be understood as any constructive element as part of the medical device to be implanted in an individual and which allows or facilitates the implantation and positioning, e.g., by making contact with or within a body or tissue part of the individual. The locator may be designed as is appropriate under the circumstances which will be described in more detail below.

“Locator probe” may form part of or be used together with a locator means and it may facilitate the correct positioning of the prosthesis at the target site in the individual. For easier visualization an opaque marker may be used.

A “valve component” in the sense of the disclosure is a biological or synthetic valve placed within the stent component and which may replace the endogenous valve function. It may comprise additional components to optimize the valve and overall prosthesis function, including, but not limited to, internal and/or external covers of the same or different biological and/or synthetic materials and sealing means.

A “sealing means” in the sense of the disclosure is a particular tissue, lining, covering, band made of synthetic and/or biological material that may be positioned outside the stent component, e.g., which may serve the purpose to prevent reflux of blood when the valve is in its closed position. In some embodiments, it is designed as a band around the stent component in e.g., an aortic valve as a symmetrical band at the location of the annular ring and which represents a sealing between the prosthesis and the endogenous valve. The “sealing means” of the disclosed prosthesis may be symmetrical and/or non-symmetrical and it may follow in particular at the outside of the prosthesis and may represent a covering. In a non-symmetrical design of the prosthesis as disclosed the “sealing means” may be more distal in the NCS section and more distal in the other two sections (16) (18).

The “target site” in the sense of the disclosure is the endogenous heart valve to be replaced by the replacement heart valve. In particular the “target site” is the position where the replacement heart valve will be implanted.

“Shortest distance” in the sense of the disclosure refers to two points that relate to design features of the prosthesis like locator and distal end which can be compared to the distance of other design features wherein the distance is measured in the same manner. “Opaque marker” in the sense of the disclosure is to be understood as any material that can be visualized by an apparatus to visualize the position of the device outside the individual's body during surgery or thereafter.

“Visualization” or “to visualize” in the sense of the disclosure includes any way to project the opaque marker and thus prosthesis position outside the individual's body.

Any other terms used in the following will be understood by the skilled person in the art and in the usual sense and manner usually applied in the art.

In the following various embodiments will be described wherein the skilled reader will understand that all features described therein may represent one single feature of the prosthesis and/or all features of the prosthesis, and any features as described herein may as well be combined in any way even if not explicitly so mentioned in the following.

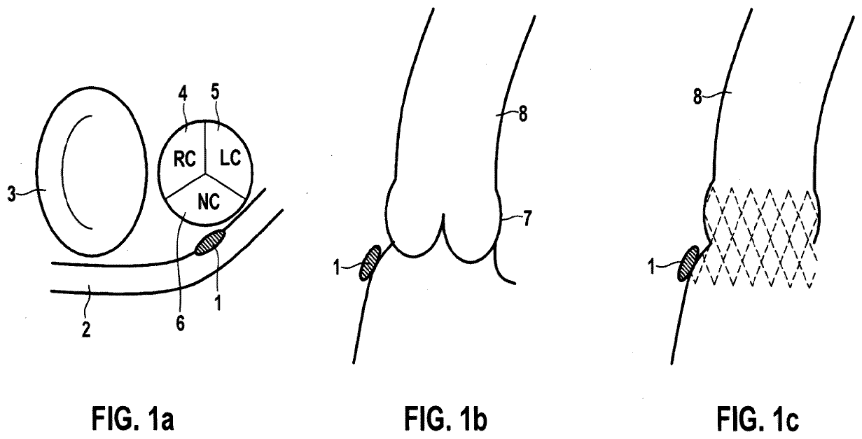

In one aspect the disclosure relates to an aortic heart valve prosthesis for reducing the need for pacemaker implantation. FIG. 1 illustrates the anatomical location of the bundle of His of the conductive system. The bundle of His is located at the septum approximately 2 mm-10 mm below the aortic annulus and the non-coronary sinus. Many transcatheter prostheses for aortic valve replacement include a balloon or self-expanded stent scaffold that anchors the prosthesis in the aortic annulus. There is evidence that the stent scaffold interferes with the conductive system of the heart, which may result in the need for pacemaker implantation. A stent scaffold that extends into the left ventricle or excessively stretches the tissue in the aortic annulus may injure or irritate the bundle of His.

One potential strategy to mitigate the risk of irritating the conductive system is to place the proximal end of the stent scaffold within the aortic annulus and avoid extension of the stent scaffold into the left ventricle. This may require accurate axial placement of the stent scaffold inside the annulus. In one aspect of the present disclosure, the stent scaffold of the valve prosthesis includes axially extending locators. The locators may be positioned within the cusp of the native aortic valve. Placement of the locators within the cusps may prevent further proximal movement of the stent scaffold into the left ventricle. By adjusting the location of the proximal end of the locators with respect to the proximal end of the stent scaffold, infra-annular placement of the stent scaffold in the aortic annulus may be assured. The distance from the proximal end of the locators to the proximal end of the stent scaffold may be less than 10 mm, for example between 1 mm-5 mm.

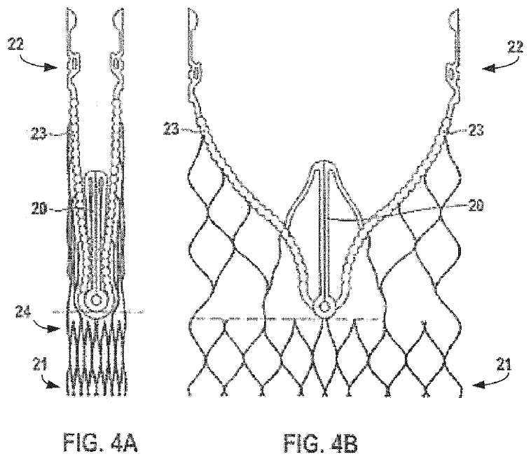

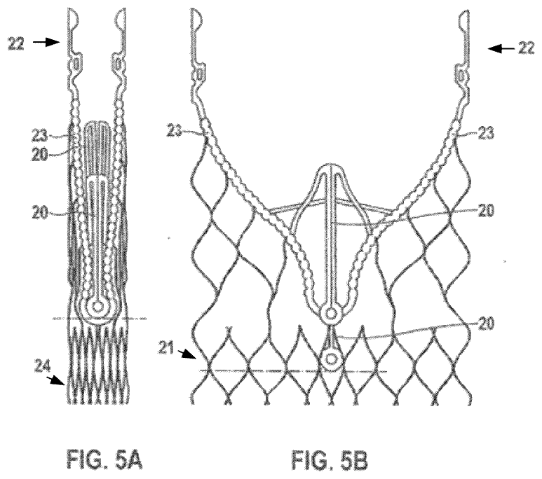

The location of the proximal end of the locators with respect to the proximal stent scaffold may require the locators to overlap with the proximal stent ring of the stent scaffold. In an exemplary embodiment, the locators and the stent scaffold of the prosthesis are cut from the same metal tubing. This may minimize the profile of the prosthesis. FIGS. 4a-4b and 5a-5b demonstrate embodiments of one-piece stent scaffolds with locators. The locators may be connected to the mid-section of the stent by diagonal struts. Expansion of the stent scaffold from the crimped configuration into the implant configuration may cause foreshortening of the diagonal struts and proximal movement of the proximal end of the locators. In the crimped configuration, the proximal end of the locators may not overlap with the most proximal stent scaffold ring. In this configuration, the stent scaffold may have a low profile for placement in the delivery system. In the expanded configuration, the proximal end of the locators may overlap with the most proximal stent scaffold ring. In some embodiments, the proximal end of the locators is located less than 10 mm, e.g., between 1 mm and 5 mm, away from the proximal end of the stent scaffold when the stent scaffold is fully expanded.

In another aspect of the disclosure, interference of the stent scaffold with the bundle of His may be mitigated by an asymmetric stent scaffold design. The most proximal segment of the stent scaffold along the non-coronary sinus may be moved distally with respect to the most proximal segments of the stent scaffold along the left and right coronary sinus. The most proximal segment of the stent scaffold along the coronary sinus may be within or supra to the aortic annulus. In conjunction with the more distal placement of the non-coronary segment of the stent scaffold, the para-valvular seal zone in the non-coronary segment of the prosthesis may extend into the non-coronary sinus. Distal extension of the seal zone may be possible since the non-coronary sinus is void of coronary arteries that need to be kept patent to perfuse the heart. Thus, an asymmetric design of the prosthesis may take advantage of the unique anatomical location of the bundle of His with respect to the annulus and the non-coronary sinus. The seal elements of the prosthesis may be located proximal to the coronary arteries in the left and right coronary sinus and distal to the coronary arteries in the non-coronary sinus.

In another aspect of the disclosure, the prosthesis may have an asymmetric design and two locators for placement in the right and left coronary cusps. The non-coronary segment of the stent scaffold may not have a locator but a supra-annular stent segment that contacts the wall of the non-coronary sinus.

In one aspect the disclosure relates to a heart valve prosthesis for reducing the need for pacemaker after positioning at a target site, comprising a stent component, a valve component, a sealing means, and at least one locator means for a defined positioning of the prosthesis at the target site of an endogenous heart valve, and wherein the prosthesis may be expandable from a non-expanded to an expanded state, and wherein in the expanded state a shortest distance between a proximal end of the locator means and a proximal end of the prosthesis may be less than 15 mm, such as less than 10, 8, or 5 mm.

The prosthesis as disclosed herein may exhibit a number of advantages compared to other devices at least partially due to its engineering. For example, one advantage may be that the design of the prosthesis makes sure that the coronary arteries are substantially not covered or blinded by any prosthesis section or area and thus the circulation of blood is not affected.

Another advantage of the prosthesis as disclosed may be that its implantation may not substantially interfere with the heart functions. For example, its implantation may result in low side effects, e.g., such that the rate of pacemakers needed may be comparably low as compared to other devices.

In one embodiment the heart valve prosthesis as disclosed comprises a locator means comprising a locator probe for the visualization of the locator means.

The prosthesis can be a tube and/or mesh like design with symmetrical end portions. It can as well have in its structure within the tube structure asymmetrical with meander like structures and it can as well be designed so that the distance between a proximal end of the locator means in case there are two or three locator means referring to the three sections as described herein and a proximal end of the prosthesis is varying in circumferential direction. The same is possible for the distal end of the prosthesis. Such a design may be suitable, for example, wherein the proximal and/or distal end is varying in its end dimensions. Such a design may provide an advantage wherein critical areas and/or various other areas of the heart may be kept without contact with the prosthesis, or the contact may be minimal or such areas of the heart even repeatedly with or without contact with the prosthesis. Thus, in some embodiments, the disclosure may allow for the respective functional areas of the heart to exhibit without interference its functions. Examples may include the coronary arteries and the bundle of His.

The prosthesis as disclosed herein in one aspect may be designed wherein in the non-expanded state the locator means and the stent component extend along a tube perimeter and in the expanded state the locator means extend at least partially outside an expanded tube perimeter.

In a prosthesis as disclosed herein which exhibits locator means in one aspect may be characterized in that in the expanded state the locator means is positioned in proximal direction at least partially over the remaining stent portion (e.g., foreshortening).

The foreshortening may allow for a design—possibly in combination with one or more other dimensions of the prosthesis—which finally allows for a precise and correct positioning of the prosthesis at the target site and may reduce—possibly together with one or more other design features of the prosthesis as disclosed herein—the need for pacemaker implantation.

In an exemplary embodiment the foreshortening of the locator means in the expanded state compared to the non-expanded state is 1, 2, 3, 4, or 5 mm. The foreshortening can be adapted in particular prosthesis seizes, e.g., 23, 25 or 27 French, as may be useful in connection with the other prosthesis design features and seizes and dimensions. In such a manner the positive effect of reduced pacemaker need may be optimized as will be appreciated by the skilled person.

The locator means may be made as a locator arch and may be attached to or forming an integral part of the stent component. In some embodiments, the prosthesis may contain three locator means, each one being positioned in one section of the prosthesis. It may as well be designed in other geometrical forms. The locator arch may be attached or forming an integral part with each of its ends with one fastening arch of the prosthesis. When the prosthesis expands from is non-expanded to its expanded stage at least two fastening arches, e.g., six fastening arches, two in each of the three prosthesis sections and three locator means respectively, may separate from each other and the locator arch may move with its tip in direction to the proximal end of the prosthesis. In this manner the positioning of the prosthesis and the dimensions of cusp positioning of the locator means, which may be one, two or three, and the proximal end within the target site (i.e. the endogenous heart valve) may be defined as well as the distances between the locator means ends as well as the proximal end of the prosthesis.

In one embodiment the fastening arch comprises fastening means which may serve for adjusting the valve component on the stent component. Other components like covers inside and/or outside the stent component made from biological or synthetic materials may also form part of the prosthesis as desired. Such covers may serve as sealing means.

In one embodiment the prosthesis as described herein is designed in a manner to substantially not cover the coronary arteries in the expanded state when placed at the target site. Thus the stent and covering components may be designed so that the respective parts are not at all covered, or exhibit one or more indentations provide for no or less or repeatedly no contact in line with the repeating heartbeat of the individual. In such a design advantageously the respective functional areas of the heart may perform their functions without that the implanted prosthesis interferes therewith.

In one embodiment the prosthesis as disclosed herein is exhibiting or can be structured in three sections and wherein one section corresponds to the right coronary sinus (RCS), a second section corresponds to the left coronary sinus (LCS) and a third section corresponds to the non-coronary coronary sinus (NCS). The prosthesis as disclosed herein can further exhibit in one embodiment the sections wherein the three sections each comprise a distal and a proximal end, and said proximal ends extend with an equal length so that the sections RCS, LCS, NCS end at the same level, or the proximal ends corresponding to the RCS and LCS sections are shorter than the NCS section.

Accordingly, in the first above alternative the end of the prosthesis in combination with the dimensions as chosen for the locator means lead to a proximal end of the prosthesis that enters the left ventricle beyond the annular ring with less than 10 mm, such as less than 5 mm. The design of the disclosed prosthesis may provide that the heart functions are not or only minimally interfered with. In the second above alternative the prosthesis may exhibit a shortened NCS section at the proximal end and thus may avoid contact with the bundle of His.

In a third alternative the prosthesis as disclosed herein may be characterized in that the three proximal sections extend with an equal length so that the sections RCS, LCS, NCS end at the same level within the left ventricle and at the same time the NCS proximal section comprises an indentation. The indentation may provide also a design feature that avoids interference of the prosthesis with the endogenous heart functions such as inter alia a regulated and repeated heart beat.

Thus all three alternative designs of the prosthesis as disclosed herein may provide for a heart valve replacement therapy with less interference of the implanted prosthesis with the endogenous heart functions and may provide for inter alia a reduced need of pacemaker implantation.

The prosthesis as disclosed herein may achieve positive and advantageous pacemaker rates, e.g., depending on the particular design features. The prosthesis as disclosed herein after implantation in an individual may thus achieve positive pacemaker rates and may induce the need for pacemaker implantation of less than 15%, such as less than 10%, e.g., less than 8%.

That the present disclosure includes a replacement heart valve prosthesis design wherein the proximal end of the proximal three sections has a shorter section NCS (17) (thus having a non-symmetrical overall design) and a non-symmetrical sealing means, which performs a good valve function and at the same time exhibits a sufficiently good sealing function and provides for a reduced need of pacemaker implantation.

In such an embodiment the sealing means may have a wave-like or U- or inverted V-shape and the sealing function may be achieved in the sections 16 and 18 at a more proximal and in the section 17 at a more distal area of the prosthesis. The areas which connect or lay between the actual sections 16, 17, 18 may be sufficient to provide for a sufficient and good sealing function.

The sealing means and sealing function may be equally designed and achieved as described above in the exemplary embodiment with an indentation area as described herein.

In another embodiment the prosthesis as disclosed may comprise a means for visualizing the positioning of the prosthesis at a target site of an individual wherein the means consists of or comprises a deformable indicator means.

The indicator means may be adapted to the other components of the prosthesis and can be adapted in any manner so that it can exhibit its function. In an embodiment the indicator means may comprise or consist of one or more wires or antennas. For visualization the indicator means may comprise radiopaque material.

In an alternative embodiment the prosthesis as described herein may comprise a counterpart to the indicator means suitable to contact each other. This counterpart may be designed in any suitable manner, and wherein the counterpart may be a locator means, a feeler, a cusp positioner, a hook and/or a rim, such as wherein the locator means, feeler, cusp positioner, hook and/or rim has U, V, Y, M or W shape.

The prosthesis as described herein may be characterized in that the indicator means and the locator means, feeler, cusp positioner, hook and/or rim may comprise the same or different materials. In one embodiment also the counterpart may comprise radiopaque material.

In at least one embodiment, a feeler means or the like may form part of the prosthesis. In one embodiment the indicator means and the feeler means or the like for a visualization means both may produce a visualizable signal. Accordingly, the operator may recognize the two signals produced by the indicator and feeler means, e.g., when the prosthesis has not reached the appropriate position. When the feeler(s), e.g., three feelers, have reached the correct position within the valve cusps the contact of the indicator means with the cusp bottom may effect a change of the geometry of the indicator means and the indicator means and the feelers may be in close proximity or in contact with each other so that the two visual signals may unify to produce at least partially at the indicator and feeler means one single signal. When the prosthesis thus has reached the correct position in the context of an aortic heart valve replacement procedure the feeler(s), e.g., the three feelers, and indicator means may be located in the cusps of the endogenous valve cusps and may produce three instead of six visual signals readily visible by the operator by way of suitable visualization means. Accordingly, the prosthesis may be positioned and its positioning can be controlled easily and efficiently.

The prosthesis as described may comprise and be made of various materials suitable for heart valve prostheses, which may consist of or comprise nitinol, soft fabric, textile, mammalian tissue, or one or more polymers, such as silicone, polyurethane, or ePTFE.

The prosthesis as described herein may be capable of replacement of any endogenous heart valve. In particular it may be useful for the replacement therapy of an aortic, or mitral heart valve.

In another aspect the disclosure relates to a method for visualizing the positioning of a prosthesis as disclosed herein wherein i. the prosthesis is delivered by appropriate means close or relatively close to the target implantation site; ii. the prosthesis is approached to its final target site; iii. the movement of the prosthesis is stopped when the deformable indicator means indicate contact with the tissue of the final target site; and the prosthesis is fully deployed at its final target site.

In yet another aspect the disclosure relates to a method for implantation of a heart valve prosthesis to a target site of an individual using a suitable catheter means and a heart valve prosthesis as described herein.

The prostheses as described herein may exhibit one or a number of advantages.

Known implants result in a pacemaker need of about 15 to 30%. In some embodiments of the present disclosure, the prosthesis is designed to reduce the need for pacemaker implantation after aortic heart valve replacement therapy by increasing the distance from the stent scaffold to the bundle of His. The corresponding need for pacemaker implantation may be less than 15%, such as less than 10% or even less than 8%.

Moreover, in embodiments wherein the design may exhibit a non-symmetric geometry in terms of the stent and the sealing material, the prosthesis may show little or no leakage, which may be unexpected for a non-symmetric design. One advantageous feature may be that in the area of the NC either the proximal section is shortened vis-à-vis the RC and LC section or the proximal part of the NC may be characterized by an indentation in direction towards the inner area of the prosthesis.

Various advantages of prosthesis as disclosed may be at least partly achieved by the design, features, and/or placement of locators, which may provide for a secure and/or precise positioning at the target site, e.g., in an advantageous manner such that they make sure that endogenous functions of the heart are satisfactory, like coronary artery function, bundle of His function, the valve as such with regard to functionality of the replacement valve, and the issue of leakage are met.

In particular the design and functionality of the locators, which may be, e.g., one, two, three or more locators, in the prosthesis and the foreshortening and the designed dimensions of the three sections of the prosthesis in relation to each other, the dimensions of the foreshortening as such and the dimensions of the proximal part of the prosthesis as well as the symmetry may contribute to various advantageous functional characteristics of the prosthesis as disclosed herein.

The attachment or design of the locators in an exemplary embodiment may be chosen to be in the middle area of the stent component. In addition the design of the locator, e.g., as an arch, may provide for a foreshortening that may be advantageous in view of a precise and proper positioning of the prosthesis.

The indicator means may be positioned or connected with the medical device in a manner so that it may make contact with the appropriate body compartment(s) or body part(s) during the implantation procedure so as to indicate correct and precise positioning of the medical device. It may comprise or consist of one or more wires or antennas.

In one embodiment the disclosure includes an indicator means and a so called counterpart means wherein these two parts are designed to be capable to contact each other.

In some embodiments, the prosthesis as described herein inter alia may be useful and may facilitate the correct positioning of a prosthesis at the target site and may avoid misplacement, e.g., based solely upon tactile feedback.

In one embodiment the prosthesis as disclosed comprises i. a shortened proximal section 17 or ii. it exhibits an indentation or open area in the proximal area of section 17, iii. the distal sections 16, 18 have either an open area at the areas where they are contacting the coronary arteries or the stent in this area does not contain a covering, or the distal section has a length in these sections that does not extend in its final positioning at the target site towards the coronary arteries, iv. a non-symmetrical sealing means in design version i.), v, three locator means designed as arches connected at their ends with fastening arches, and vi. a foreshortening of 5 mm.

Such an embodiment may be advantageous in terms of less interference with the endogenous heart function, may provide sufficient and/or good sealing features, and may be correctly positioned by way of minimally invasive catheter delivery.

### LIST OF REFERENCE NUMBERS

1 Bundle of His
2 Septum
3 Mitral Valve
4 leaflet (RC) of aortic valve
5 leaflet (LC) of aortic valve
6 leaflet (NC) of aortic valve
7 annulus
8 aortic arch
9 RC (right coronary) sinus
10 NC (nucleus coronary) sinus
11 LC (left coronary) sinus
12 left coronary artery
13 right coronary artery
14 aortic heart valve prosthesis
15 sealing means
16 first section of prosthesis
17 second section of prosthesis
18 third section of prosthesis
19 area without stent and sealing means or designed as indentation
20 locator means
21 proximal section (preferred as stent ring)
21′ proximal end
22 distal stent section
23 fastening arch
24 foreshortening distance/length
25 indicator means
26 positioning direction of prosthesis
27 aortic cusp
28 locator cover
29 radio-opaque marker
30 sinus of vasalva
31 calcified aortic leaflets
32 left ventricle
D distance of proximal end of locator means and proximal end of prosthesis

What is claimed:

1. A heart valve prosthesis, comprising a stent component, a valve component, a seal, and at least one locator for positioning the prosthesis at a target site of an endogenous heart valve, wherein the prosthesis is expandable from a non-expanded state to an expanded state, and wherein in the expanded state a shortest distance between a proximal end of the locator and a proximal end of the prosthesis is less than 10 mm.
